(19) United States
(12) Patent Application Publication
(10) Pub. No.: US 2010/0144009 A1
(43) Pub. Date: Jun. 10, 2010

(54) Extracting And Purifying Lipoxygenase

(75) Inventors: Michael Patane, New South Wales (AU); Akshat Talwalkar, New South Wales (AU)

Correspondence Address: Ballard Spahr LLP, SUITE 1000, 999 PEACHTREE STREET, ATLANTA, GA, 30309-3915, US

(73) Assignee: Protech Research PTY Ltd.

(21) Appl. No.: 12/520,745

(22) PCT Filed: Dec. 20, 2007
(86) PCT No.: PCT/AU07/01971

§ 371 Date: Jan. 27, 2010

(30) Foreign Application Priority Data
Dec. 20, 2006 (AU) 2006907091

(51) Int. Cl. C12N 9/02

(52) U.S. Cl. 435/189

(57) ABSTRACT

The present invention relates to the extraction and purification of lipoxygenase from plant matter, including soy beans. A process for purifying lipoxygenase from plant matter is provided.

### FIELD OF THE INVENTION

The present invention relates to the extraction and purification of lipoxygenase from plant matter, including soy beans.

### BACKGROUND OF THE INVENTION

Lipoxygenase (also known as lipoxidase, lipoperoxidase, and carotene oxidase) (EC1.13.11.12) catalyse the oxidation of unsaturated fatty acids in lipids containing cis-1-4-pentadiene system by molecular oxygen to hydroperoxides. Linolenic and linoleic acids are the most common fatty acid moieties in unsaturated lipids that act as substrates. Accordingly, one reaction catalysed by lipoxygenase is:

linoleate+O(2) [symbol] (9Z,11E)-(13S)-13-hydroperoxyoctadeca-9,11-dienoate.

Lipoxygenase also oxidises other methylene-interrupted polyunsaturated fatty acids.

Lipoxygenase is found in plants including soy beans and other beans such as navy beans, kidney beans and lima beans. It is also found in common legumes i.e. beans, peas, lentils, etc. It is also found in potato, horse radish, turnips, and other tubers. Various types of seeds, bulbs and nuts also contain lipoxygenase.

Three lipoxygenase isozymes, LOX1, LOX2, and LOX3 (accession no.s P08170, P09439 and P09186 respectively) have been found in soy bean cotyledons. Other forms, LOX4 (P38417) and LOXX (P24095), have also been observed.

Lipoxygenase as found in soy flour is used in the baking industry to improve dough and crumb quality and to bleach pigments in the dough during breadmaking. In these applications, lipoxygenase reduces the binding of added shortening with protein during dough mixing, increases gluten strength, and improves baking performance and product quality.

Typically, the lipoxygenase is provided to the dough in the form of enzyme active soy flour. It is destroyed during the baking process by thermally-induced denaturation.

A number of groups have sought to purify lipoxygenase. Each of these groups has taken the approach of using de-fatted soy bean flour as a starting material. An aqueous suspension is prepared in the form of a slurry. After removing solids from the solution, ammonium sulphate is added to precipitate proteins and hence to concentrate lipoxygenase. The ammonium sulphate must then be removed from the concentrated lipoxygenase-protein matrix. In some circumstances, this removal of ammonium sulphate requires dialysis. In other circumstances, more specialised chromatography is required. Consequently, many processes for purifying lipoxygenase from ancillary proteins have limited commercial application.

Further, the problem with each these approaches is that they have a relatively poor yield and purification efficiency.

### SUMMARY OF THE INVENTION

The invention seeks to minimise or reduce the above limitations or problems and in certain embodiments to provide a process for producing a purified extract of a lipoxygenase enzyme from a plant matter. The process includes the steps of:
extracting lipoxygenase from a plant matter into an aqueous solution to form an extract;
providing the extract with a pH below the isoelectric point of lipoxygenase to selectively precipitate or flocculate compounds from the extract while retaining lipoxygenase in solution, to form an acidified extract;
separating the precipitated or flocculated compounds from the acidified extract, to provide a purified extract of a lipoxygenase.

### DETAILED DESCRIPTION OF THE EMBODIMENTS

The inventor has found that by using plant matter as a starting material for the purification of a lipoxygenase, a much greater yield of lipoxygenase can be obtained. In more detail, as described further, a yield of 3 to 4 times more enzyme could be obtained from soy beans extracted according to the process described herein as compared with enzyme obtained from an extraction involving enzyme active soy flour.

A total purification factor approaching 125 fold was obtained when the extract obtained by the process described herein was further subjected to ion exchange chromatography and ultrafiltration.

One particular advantage is that according to the process described herein, the specific activity of the lipoxygenase can be increased and hence the enzyme enriched in the extract without having to use an ammonium sulphate concentration step.

Thus in certain embodiments there is provided a process for purifying a lipoxygenase from a plant matter including the steps of:
extracting lipoxygenase from a plant matter into an aqueous solution to form an extract;
providing the extract with a pH below the isoelectric point of lipoxygenase to selectively precipitate compounds from the extract while retaining lipoxygenase in solution, to form an acidified extract;
separating the precipitated compounds from the acidified extract, to provide a purified extract of lipoxygenase.

According to this embodiment, in the first step, lipoxygenase is extracted from the plant matter into the aqueous solution. Water is one example of an aqueous solution. Other solutions, for example salt solutions or pH adjusted solutions that facilitate the transfer of the lipoxygenase from the plant matter into the solution may also be used.

In certain embodiments, the transfer of the lipoxygenase into the solution is facilitated by applying a chemical or physical treatment to the plant matter that facilitates ingress of the aqueous solution into the plant matter. With this treatment, the tissue of the matter is made vulnerable or otherwise exposed to the solution. Accordingly, tissue that would otherwise be less readily hydrated by the solution becomes hydrated, resulting in an increase in the amount of enzyme that may be extracted from the plant matter.

One particularly useful treatment where the enzyme is to be extracted from plant matter including cotyledons, for example a bean, such as a soy bean, or a legume, or a cereal such as barley, is a milling process in which the matter is milled to create cracks or fissures in the periphery of the tissue or to produce a milled flour through which the solution may seep to hydrate the matter. Other physical treatments known to the skilled worker may also be applied. Whether these are applicable will depend largely on the physical characteristics of the plant matter to be treated and taking account one objective of the first step of the process which is to extract as much enzyme from the plant matter into the solution as is required by the particular application.

Thus in one embodiment, the extract is formed from milling the plant matter to facilitate ingress of the aqueous solution into the plant matter. This produces a hydrated slurry.

One way of facilitating transfer of the enzyme into the solution is to steep the plant matter in the aqueous solution to form a steep solution. The amount of time required for steeping depends again on the physical characteristics of the plant matter to be treated and the need to extract as much enzyme from the plant matter into the solution as is required by the particular application. Steeping times may be as little as 1 hour for some plant matter. Where the plant matter is a bean or legume, the steeping time is about 6-30 hours. Longer times may be used although this may require providing conditions to prevent microbial contamination or spoilage. In certain embodiments, at the completion of the steeping step, the solution has generally absorbed or seeped or soaked into the plant matter leaving it generally hydrated throughout. Thus in one embodiment, the plant matter is steeped in the aqueous solution to form a steep solution, before the plant matter is milled.

The inventor has found that the amount of enzyme extracted from the plant matter can be substantially increased when a subsequent physical treatment described above is applied to the steeped plant matter in the presence of the steep solution. One advantage is that the extraction efficiency of the process may be increased. Another is that the production costs are decreased by minimising water usage. Thus in one embodiment, the plant matter is milled in the steep solution.

During the formation of the extract according to the first step of the process described above, the aqueous solution, whether a “steeped solution” or otherwise, may be provided with an alkaline pH, for example a pH of about 7 to 9. The inventor has found that this is useful for further facilitating the extraction of the lipoxygenase into the solution. In this embodiment, the plant material may be maintained in the aqueous solution for about 1 to 30 hours. Shorter or longer times are possible. Thus in one embodiment, the extract is provided with an alkaline pH and milled to facilitate extraction of the lipoxygenase into the solution.

In some embodiments, and in particular those where a chemical or physical treatment as described above has been applied to the plant matter during the extraction process, particulate matter in the form of cellular or tissue debris may become dispersed or otherwise contaminate the solution. In these embodiments, it may be necessary to remove the particulate matter from the aqueous solution to form the extract. As described herein, this can be done by filtering the solution to remove the particulate material, prior to forming the extract. Thus in one embodiment, the process includes filtering the solution to remove cellular material from the solution, to form the extract.

Further, in some embodiments it may be advantageous to provide a normal pH to the solution and to heat it before the extract is formed. These steps facilitate the separation of compounds in the extract, such as proteins and the like, from lipoxygenase. Where the solution has previously been provided with an alkaline pH as described above, in these embodiments it is reduced from the alkaline pH 9.0 to about pH 7.0. Further, the solution is heated to about 55° C. to form the extract. Thus in one embodiment the process includes heating and reducing the pH of the solution to separate compounds in the solution from lipoxygenase, to form the extract.

In a second step, the process of the invention includes forming an acidified extract from the extract formed in the first step of the process. This is done by providing a pH below the isoelectric point of lipoxygenase to the extract to selectively precipitate compounds from the extract while retaining lipoxygenase in solution. One objective of this step is to selectively purify the lipoxygenase in solution from other compounds, especially proteins in the solution by causing the latter to aggregate or to flocculate and so to precipitate out of solution, leaving the lipoxygenase in solution. This step is based on the phenomenon known as “isoelectric precipitation” or “fractional precipitation” wherein a protein species is observed to fall out of solution at a given pH (its isoelectric point) at which it has no net charge.

The isoelectric point of LOX1 is about 5.8, LOX2 about 6.2 and LOX3 about 6.3. Typically, the pH of the extract is adjusted to about pH 4.5 to form the acidified extract. However, higher or lower pH conditions could be applied depending on the isozyme to be purified and the isoelectric point or “pl” of the contaminant proteins in the extract prior to acidification.

Typically the extract is heated, for example to about 55° C. as it is acidified to pH 4.5 to form the acidified extract.

In some embodiments, the pH of the solution may be ramped down in a step wise manner to gradually cause precipitation of contaminant proteins using citric acid or glucono delta lactone or “GDL”.

The precipitated proteins may be separated from the lipoxygenase in the acidified extract by a number of approaches. In one embodiment, the acidified extract is allowed to cool, or otherwise chill, and the precipitated protein allowed to settle, after which the acidified extract is removed, for example, by siphoning, to separate the extract from the precipitated protein. In this example it has been found that allowing the acidified extract to settle for about 24 hours at about 10° C. is useful for causing the precipitated protein to settle. Thus in one embodiment, the acidified extract is chilled to facilitate separation of the precipitated compounds from the extract, hence providing the purified extract.

In a preferred embodiment, the flocculated protein is separated by a filtration process at 55° C. as an enhancement to chilling to improve purification of the enzyme. This process provides for a large agglomeration of proteins more easily separated by filtration.

After the separation of the precipitated proteins from the acidified extract, the pH of the acidified extract may be adjusted back to alkaline conditions, for example, to about pH 9 to provide an enhanced activity of the enzyme.

The extract may then be further processed to concentrate the lipoxygenase, selectively removing solvent from the purified extract to increase the concentration of the lipoxygenase in the purified extract. This can be done by a number of approaches including precipitation and re-dissolution, chromatography etc. However, the inventor has found microfiltration, ion exchange and ultrafiltration to be a particularly useful approach.

Thus in one embodiment, the process includes selectively removing solvent from the purified extract to isolate and increase the concentration of the lipoxygenase in the purified extract.

### EXAMPLES

### Example 1

### Materials and Methods

### Enzyme Assay:

Based on the methylene blue bleaching method described by Suda et al (1995).

### Substrate Preparation:

70 mg of linoleic acid (Sigma) and 70 mg of Tween20 was homogenized in 4 mL DW with a Pasteur pipette. To obtain a clear solution, 0.55 mL 0.5N NaOH was added and the solution was made up to 25 mL total volume with DW. 1.5 mL aliquots of this substrate solution was placed into vials, covered with N₂ gas and stored in the freezer until use.

Enzyme assay: The reaction mixture contained 0.7 mL 0.2 M Tris-HCl buffer, pH 9.0, 0.1 mL 100 μM methylene blue, 0.1 mL 10 mM sodium linoleate substrate and 0.1 mL of soybean extract sample. The reaction was initiated by the addition of the soybean sample and the decrease in absorbance at 660 nm was recorded on the spectrophotometer.

### Example 2

### Activity of Lipoxygenase Obtained from Soy Flour as Compared with Soy Bean

Full fat enzyme active soy flour was prepared as a 1:5 w/v suspension at pH 9.0 (0.1M Tris HCL) with 0.1% w/v surfactant (Triton-X-100) and processed for 6 hours. The enzyme activity at this time was found to be 45 units.

Soy beans by comparison were prepared as a 1:5 w/v suspension and soaked at pH 9.0 (0.1M Tris HCL) with 0.1% w/v surfactant (Triton-X-100) for 6-24 hours and processed using a colloid mill incorporating the steep water during the milling process.

The activity comparisons are set out in the table below.

| Materials | Units |
|---|---|
| Soy Beans | 175 |
| Soy Flour | 45 |

One unit of lipoxygenase activity is the amount of enzyme required to decrease the absorbance of methylene blue in the defined assay conditions by 0.001 absorbance units at 660 nm in 1 minute at 25° C.

It was further found that by soaking soy beans for 6-24 hours and their subsequent wet milling with the steep water was found to give up to 4 times more activity than extracting enzyme from soy flour, presumably because of the better hydration of the bean when soaked, which could allow easier and better extraction of lipoxygenase.

### Example 3

### Exemplary Flow Chart for Purification of Lipoxygenase from Soy Bean or Soy Flour

### Example 4

### Results

TABLE 1
Fold purification for lipoxygenase

| | Activity | Protein | Specific | |
|---|---|---|---|---|
| Sample | U/ml | (mg/mL) | Activity | Fold purification |
| Crude | 97 | 132.5 | 0.73 | 1 |
| Acidified | 162 | 13.97 | 11.59 | 15.02 |
| Post Heat | 194 | 13.75 | 14.11 | 19.12 |
| Ion exchange | 114 | 1.41 | 80.85 | 110.65 |
| Concentration | 989 | 10.9 | 90.73 | 123.90 |

TABLE 2
Lipoxygenase activity at differental pH

| | pH | | | | |
|---|---|---|---|---|---|
| | 5 | 6 | 7 | 8 | 9 |
| activity | 31 | 41 | 59 | 64 | 87 |
| U/ml | | | | | |

Thus the optimum pH for assay and extraction is pH 9

TABLE 3
Activity of lipoxygenase post heating the acidified extract

| | pH | | | | |
|---|---|---|---|---|---|
| | 5 | 6 | 7 | 8 | 9 |
| activity | 74 | 124 | 185 | 89 | 24 |
| U/ml | | | | | |

Thus the optimum pH for heating the post acidified enzyme is pH 7

TABLE 4
Activity of lipoxygenase at different temperatures

| | Temperature | | | | |
|---|---|---|---|---|---|
| | 30 | 40 | 50* | 60* | 70 |
| activity | 177 | 179 | 185 | 112 | 8 |
| U/ml | | | | | |

*optimum heat stability temperature was 55° C.

TABLE 5
The effect of cations on activity of Lipoxygenase LOX-1
10 mL of LOX1 containing 50 mM CaCl₂, MgCl, ZnOAc, Fe³⁺Cl
was incubated for 1 hour at 55° C.

| Cation | Control | Ca | Mg | Zn | Fe |
|---|---|---|---|---|---|
| activity | 0181 | 125 | 167 | 94 | 0 |
| U/ml | | | | | |

Magnesium and calcium ions can be used to enhance enzyme activity.

### Example 5

### Process at Pilot Scale

### Soaking of Soy Bean

100 kg of Cowrie soy bean are soaked for 24 hours in 400 L of water adjusted to pH 9.0.

### Milling

Once the soy beans have been soaked for up to 24 hours, the beans are removed from the buffer. The beans and buffer are then fed into the colloid mill at a ratio of 1:4 (beans to buffer) at a flow rate of 3 L/min buffer. There needs to be a steady flow of liquid into the colloid mill to minimise heat build up in the extract.

### Extraction

pH of the milled extract is maintained at pH 9.0 to maximise extraction from plant material (also this is optimum pH for LOX1 activity). The macerated soy bean extract can be further held at this pH for 1 hour to improve extraction of LOX1 from the bean extract.

### Separation of Cellular Debris

The crude extract is passed through a 3 tiered vibrating screen using a diaphragm pump.

The flow rate is controlled so there is no excess build up of unwanted cellular material on the 500 μm top screen. The three mesh screens that the extract passes through are 500 μm, 250 μm and 100 μm.

Cellular material is removed by the vibration of the screen that permits liquid to pass through the screen while solid material above the cut off is retained and removed by the solids spout.

Once the liquid is passed through the vibrating screen, it is collected and transferred to a mixer for the heating and acidification step. pH is adjusted to pH 7.0 for the heating step.

### Heating and Acidification

The parameters for heating are set at 55° C. Once this temperature is achieved, the extract is maintained at this temperature for 15 min, the extract is then acidified to pH 4.5 using 1 M citric acid. Alternatively, known amounts of freshly made GDL solution can be mixed with the extract prior to heating with the pH drop due to conversion of GDL to gluconic acid is monitored.

When pH reaches 4.5, most of the soy protein (glycinin) flocculates and separates from the whey. The liquid extract is later chilled to <10° C. (recent trials have been successful in removing a large amount of flocculated protein while it is held at 55° C. while passing it through a 40 μm screen).

### Chilling

The chilled extract is either passed through a heat exchanger or transferred to a conical tank and held for 24 hours in a cold room at <10° C. for settling of the soy protein isolate. After the settling has occurred the clarified extract is siphoned from the top and removed. The bottom half of the extract is filtered through a 40 μm mesh screen then centrifuged to remove unwanted protein.

The clarified extract is then adjusted to pH 9.0 for further filtration while providing maximum enzyme activity

### Microfiltration

The extract is microfiltered through a 0.45 μm membrane and the permeate collected.

### Ion Exchange

Lipoxygenase may be isolated by DEAE anionic exchange chromatography using 25 mM Tris-HCL (pH 7.0) at a flow rate of 3 mL per minute on a 50 mLI column and eluted with 0.5 M sodium chloride.

### Ultrafiltration

The extract is concentrated using a 10 kDa NMWC (concentrated 10-30 fold its original volume).

It will be understood that the invention disclosed and defined in this specification extends to all alternative combinations of two or more of the individual features mentioned or evident from the text or drawings. All of these different combinations constitute various alternative aspects of the invention.

1. A process for purifying lipoxygenase from plant matter including the steps of:
extracting lipoxygenase from a plant matter into an aqueous solution to form an extract;
providing the extract with a pH below the isoelectric point of lipoxygenase to selectively precipitate compounds from the extract while retaining lipoxygenase in solution, to form an acidified extract;
separating the precipitated compounds from the acidified extract, to provide a purified extract of lipoxygenase.

2. The process according to claim 1 wherein the extract is formed from milled and hydrated plant matter to facilitate ingress of the aqueous solution into the plant matter.

3. The process according to claim 2 wherein the plant matter is steeped in the aqueous solution to form a steep solution, before the plant matter is milled.

4. The process according to claim 3 wherein the plant matter is milled in the steep solution.

5. The process according to claim 2 wherein the solution is provided with an alkaline pH before and after milling to facilitate extraction of the lipoxygenase into the solution.

6. The process according to claim 1 including filtering the solution to remove cellular material from the solution, to form the extract.

7. The process according to claim 1 including heating and reducing the pH of the solution to separate compounds in the solution from lipoxygenase, to form the extract.

8. The process according to claim 1 including filtering the extract above 55° C. and then chilling the acidified extract to facilitate separation of the precipitated compounds from the extract.

9. The process according to claim 1 including providing the acidified extract with an alkaline pH to increase the specific activity of the enzyme.

10. The process according to claim 1 including selectively removing solvent from the purified extract to increase the concentration of the lipoxygenase in the purified extract.
